(19) United States
(12) Patent Application Publication
(10) Pub. No.: US 2024/0266013 A1
(43) Pub. Date: Aug. 8, 2024

(54) SYSTEM, METHOD, AND COMPUTER-ACCESSIBLE MEDIUM FOR POINT PROCESSES FOR COMPETING OBSERVATIONS WITH RECURRENT NETWORKS

(71) Applicant: The Trustees of Columbia University in The City of New York, New York, NY (US)

(72) Inventors: Shreyas BHAVE, New York, NY (US); Adler PEROTTE, New York, NY (US)

(21) Appl. No.: 18/417,066

(22) Filed: Jan. 19, 2024

Related U.S. Application Data
(63) Continuation of application No. PCT/US2022/037832, filed on Jul. 21, 2022.
(60) Provisional application No. 63/224,238, filed on Jul. 21, 2021.
(60) Provisional application No. 63/227,647, filed on Jul. 30, 2021.

(51) Int. Cl. G16H 10/60

(52) U.S. Cl. CPC G16H 10/60

(57) ABSTRACT

Modeling exemplary EHR data can be useful in a broad range of applications including prediction of future conditions or building latent representations of patient history. Exemplary embodiments of the present disclosure can model the full longitudinal history of a patient using a generative multivariate point process that (optionally simultaneously) can, e.g., (1) model irregularly sampled events probabilistically without discretization or interpolation; (2) have a closed-form likelihood, making training straightforward; (3) encode dependence between times and events with an approach inspired by competing risk models; and (4) facilitate a direct sampling. The exemplary embodiments can provide an improved performance on next-event prediction compared to existing approaches.

### CROSS-REFERENCE TO RELATED APPLICATION(S)

This application relates to and claims priority from U.S. Patent Application Nos. 63/224,238 and 63/227,647, filed on Jul. 21, 2021 and Jul. 30, 2021, respectively, the entire disclosures of which are incorporated herein by reference.

### STATEMENT REGARDING FEDERALLY SPONSORED RESEARCH

This invention was made with government support under Grant No. T15LM007079-29 awarded by the National Library of Medicine Training. The government has certain rights in the invention.

### FIELD OF THE DISCLOSURE

The present disclosure relates generally to probabilistic framework for modeling irregularly sampled data, and more specifically, to exemplary embodiments of exemplary system, method, and computer-accessible medium for modeling EHR data as a Multivariate Temporal Point Process and detect medical conditions based on the model.

### BACKGROUND INFORMATION

Multivariate, irregularly sampled time series data are ubiquitous in many data modalities across healthcare, including principally Electronic Health Records (EHR) data. They can be defined in the context where a dataset contains a set of time series where each time series contains a sequence of pairs {(tᵢ,ϵᵢ)}ᵢ₌₁^{N} where tᵢ represents the time and eᵢ represents a particular event type. In some cases, past sequences strongly inform which events are likely to happen in the future and when. In the case of EHRs, each time series can be a longitudinal history of a patient's visits, lab tests, administration of medications, diagnoses of conditions and more. Modeling EHRs as sequences of such events and building better generative models may be considered in a wide range of applications including prediction of future events (e.g., conditions, readmission, diagnostic), building latent representations of a patient's history and generation of synthetic data.

More generally, many types of data can be represented as sequences of events and the associated times at which those events occur. These kinds of sequences are known as event streams and are common in many contexts ranging from consumer behavior in the form of sequences of online interactions to medical events often recorded in the EHR. A characteristic of such data can be that the patterns of the past sequences of events and associated times affect the likelihood of future events and times. In order to accurately predict future events and times, a model may capture these associations which may occur on short or longer timescales.

Prior generative approaches for modeling this kind of data are lacking in one or more of the following characteristics: (1) times and events may be considered conditionally independent given history which can be limiting for prediction and simulation (2) direct sampling may not be possible (3) optimization is challenging due to a lack of a closed-form likelihood. In healthcare, events and times may often be tightly linked. In particular, if the next event happens within minutes versus after many days, this can change the prediction about what the next event is.

Neural temporal point process models have garnered substantial interest in recent years with the emergence of neural density estimation approaches. These methods may employ the basic framework of a temporal point process but may differ in the following categories: (1) independence assumptions between events and times; (2) the probabilistic object which is modeled (e.g., conditional intensity function, cumulative intensity, conditional probability density); and/or (3) the approach used to encode past history to predict next event (e.g., continuous LSTM, GRU, etc.). As a result of the choices made in each of these categories, models can have different properties. Favorable properties as outlined in Shchur et al. (2019) can include, e.g., (1) a closed-form likelihood for ease of optimization; (2) direct sampling (of next event and time given history) for ease of use; and/or (3) distributional flexibility.

In one of the earliest works in neural point processes, Du et al. (2016) use a simple RNN to encode history, reading in data as tuples of times and events. They use the hidden state of the RNN hⱼ to model the conditional intensity function which has a fixed specification. With this specification, the time until next event can be a unimodal distribution. They can also model the next event as conditionally independent of next time. As such, the flexibility of the model may be restricted by the exponential specification and next time and event are not tightly coupled. Additionally, the history encoding approach may not directly handle irregular sampling.

The neural hawkes process (see Mei and Eisner, 2016) can address many of these issues. They specify a multivariate point process which may take competing risks into account. Additionally, they may employ an approach which uses a custom continuous time LSTM architecture in an attempt to better encode history. One drawback of this approach is that it chooses to model the conditional intensity function which reduces the efficiency of optimization by requiring a Monte Carlo estimate of an integral. Additionally, sampling requires a thinning algorithm.

Intensity-free temporal point processes (see Shchur et al., 2019) can take the approach of directly modeling the conditional probability of the next event time using mixture density networks, avoiding the issues that arise from modeling conditional intensities. This may allows for direct sampling and a closed-form likelihood. However, they may model times independently of events. Additionally, they may use the same architecture as Du et al. (2016) to model history which may not account for irregular sampling.

Several other methods (see Okawa et al. (2019), Omi et al. (2019), Taddy et al. (2012), and Tabibian et al. (2017)) have been proposed which use different approaches to model conditional intensity functions which may suffer from similar issues as those outlined above.

Thus, it may be beneficial to provide an exemplary system, method, and computer-accessible medium for to model EHR data as a Multivariate Temporal Point Process, a probabilistic framework for modeling irregularly sampled data, which can overcome at least some of the deficiencies described herein above.

### SUMMARY OF EXEMPLARY EMBODIMENTS

The exemplary system, method and computer-accessible medium, according to an exemplary embodiments of the present disclosure, can relate to modeling EHR data as a Multivariate Temporal Point Process, which can be an exemplary probabilistic framework for modeling irregularly sampled data. In this exemplary framework, both the time until the next event and the type of event can be modeled probabilistically by conditioning on a summary of the entire history prior to that point. The exemplary system, method and computer-accessible medium, according to an exemplary embodiments of the present disclosure, can include a multivariate model which (optionally simultaneously): (1) specifies dependence between events and times inspired by competing risks; (2) allows for direct sampling; (3) specifies a closed-form likelihood, making training efficient and stochastic optimization straightforward; and/or (4) models event stream data without imputation and/or discretization.

The exemplary system, method and computer-accessible medium, according to an exemplary embodiments of the present disclosure, can include and/or utilize a model which can be evaluated on datasets used in the point process literature: a MIMIC-II (see Johnson et al., 2016) dataset consisting of ICU visits where the events can be conditions and their timestamps and a Stack Overflow dataset which can consist of two years of data on users receiving sequences of badges on the online forum. The exemplary system, method and computer-accessible medium, according to an exemplary embodiments of the present disclosure, can be evaluated on synthetic EHR data from Synthea (see Walonoski et al., 2018) and Synthea (Ear Infection) generated in prior related work (see Enguehard et al., 2020). These datasets are publicly available, which allows for data transparency and for direct comparison to relevant prior work. The exemplary model is compared against recently proposed approaches which differ in key ways as outlined in the Related Works. The exemplary system, method and computer-accessible medium, according to an exemplary embodiments of the present disclosure, can be evaluated on both prediction of event type given next event time as well as joint probability of next event and next time on a held-out test set. The particular metrics for assessing these can be weighted F1/AUROC and negative log likelihood normalized by time respectively.

The present disclosure relates to an exemplary system, method and computer-accessible medium for predicting medical events used for a treatment of at least particular one of a plurality of patients. The exemplary system, method and computer-accessible medium, according to an exemplary embodiments of the present disclosure, can receive first medical information for each of the patients, wherein the medical information includes at least one of the medical events and a time associated with the at least one of the medical events; generate a summary of the medical information; generate a multivariate point process model based on the summarized medical information; receive second medical information for the at least particular one of the patients; and predict and facilitate at least possible one of the medical events and a predicted time of the at least possible one of the medical events for the at least particular one of the patients.

The exemplary system, method and computer-accessible medium, according to exemplary embodiments of the present disclosure, can specify a dependence between the future time and the future event.

In the exemplary system, method and computer-accessible medium, according to exemplary embodiments of the present disclosure, the multivariate point process model can specify a conditional probability of each of the medical events.

In the exemplary system, method and computer-accessible medium, according to exemplary embodiments of the present disclosure, the multivariate point process model can be based on a survival function and a history function which is associated with the summarized medical information.

In the exemplary system, method and computer-accessible medium, according to exemplary embodiments of the present disclosure, the conditional probability is determined based on the survival function in view of the history function.

In the exemplary system, method and computer-accessible medium, according to exemplary embodiments of the present disclosure, the multivariate point process model can facilitate a generation of the at least possible one of the medical events and the predicted time based on a sample from all of event distributions.

The exemplary system, method and computer-accessible medium, according to exemplary embodiments of the present disclosure, can facilitate or control the treatment of the at least particular one of the patients based on the generated at least possible one of the medical events and the predicted time.

The exemplary system, method and computer-accessible medium, according to exemplary embodiments of the present disclosure, can facilitate the model to, among others, capture long-term dependencies and (optionally) additionally simulate entire sequences of future events based on past history. The facility to efficiently simulate multiple events into the future, e.g., as presented by the exemplary system, method and computer-accessible medium, according to exemplary embodiments of the present disclosure, can create many possible use-cases for this model including applications to causal inference and reinforcement learning. In order to train and evaluate how well a model can predict multiple events sequentially, the exemplary system, method and computer-accessible medium, according to exemplary embodiments of the present disclosure, can include a loss function which can encourage the model to learn using a score gradient estimator. In order to evaluate whether the exemplary model can capture multi-step predictions, the exemplary system, method and computer-accessible medium, according to exemplary embodiments of the present disclosure, can measure whether a particular future event is accurately predicted as happening within a certain time window. Once the exemplary model can simulate multiple steps forward, this can, e.g., open the door to many possible applications. In one example, both intervention A and intervention B can be candidates to be prescribed to a patient but their comparative effectiveness may not be clear. In this example, the exemplary system, method and computer-accessible medium, according to exemplary embodiments of the present disclosure, can use a learned model to simulate forward many events into the future to determine the possible outcomes of each intervention for a specific patient. An exemplary model trained on a large set of historical data can provide a prediction as to the expected effectiveness of each intervention.

The exemplary system, method and computer-accessible medium, according to exemplary embodiments of the present disclosure, can be useful in any scenario where data is in the form of event streams (sequences of event and time pairs) where past sequences and/or patterns of events may strongly influence the likelihood of future events and their times.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 shows High Level Diagram and Associated Graphs for an exemplary model according to an exemplary embodiment of the present disclosure;

FIG. 2 shows an exemplary Multi-Channel LSTM Diagram for encoding history according to exemplary embodiments of the present disclosure;

FIG. 3 shows an exemplary diagram visualizing of AUROC by event type accordingly to an exemplary embodiment of the present disclosure;

FIG. 4 shows an exemplary diagram of synthetic data in the EHR which can be represented as a multivariate point process, according to an exemplary embodiment of the present disclosure;

FIGS. 5(a)-5(l) show an exemplary multivariate point process as it is sampled forward, according to an exemplary embodiment of the present disclosure;

FIG. 6 shows an exemplary multivariate point process which is modeled in a more traditional manner using conditional intensity functions λᵢ rather than event probabilities, according to an exemplary embodiment of the present disclosure; and

FIG. 7 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

Exemplary Generalizable Insights about Machine Learning in Context of Healthcare

Some exemplary predictive modeling approaches built on EHR longitudinal data may make simplifying assumptions either when modeling feature inputs or the output events of interest. When modeling irregularly sampled time series features, the approach may be to discretize the irregularly sampled sequence into equal bins and develop an interpolation model for data that is missing prior to using a standard approach (e.g., LSTM) for regularly sampled data (see Che et al., 2018). Such an approach may suffer from both loss of information and introduction of noise. Another criticism of many prediction models, and more specifically survival models, can be that they may not handle competing risks. Without taking competing risks into account, model estimation and prediction can be biased due to misspecification.

Exemplary embodiments of the present disclosure can model the full longitudinal history of a patient using a multivariate point process model that has, among others, several advantages: (1) Irregularly sampled events can be modeled directly without discretization or interpolation (2); A closed-form likelihood can make training straightforward; (3) The model can encode dependence between times and events with an approach inspired by competing risk; and/or (4) Direct sampling is possible. Exemplary embodiments of the present disclosure facilitate an improved performance with EHR data on next-event prediction compared to other approaches. Exemplary results provide evidence that incorporating competing risks can be considered for modeling EHR data especially in the context of next-event prediction.

The exemplary system, method and computer-accessible medium, according to an exemplary embodiments of the present disclosure, can handle the primary dependencies of EHR data. The exemplary system, method and computer-accessible medium, according to an exemplary embodiments of the present disclosure, can be a multivariate point process with dependencies between events and times, can directly model the conditional probabilities of each event given history, and can employ a multi-channel neural architecture to model the irregularly sampled signal for encoding history.

### Exemplary Temporal Point Process

A temporal point process (TPP) can be a random process which is meant to model a sequence of N times (t₀, t₁, . . . , t_{N}). Such a process is defined by specifying a distribution for the interevent times, or the times between successive events conditioned on history up until each successive point Hₜₙ₋₁. A TPP is fully specified by the joint density f(t₀, t₁, . . . , t_{N})=Πₙf(tₙ| . . . , tₙ₋₂, tₙ₋₁)=Πₙf(tₙ|Hₜ_{ₙ₋₁}). The traditional method of modeling this data is to use a conditional intensity function λ*(t)=λ_{θ}(t|H) where θ is the set of model parameters and the star denotes that the intensity is conditioned on all historical times. This intensity function describes the instantaneous rate at which an event happens given that the event hasn't happened yet:

$${λ}^{*} ( t ) = {lim}_{dt \to 0} \frac{P ( t \leq T < \text{?} + dt )}{dt * S ( t )} = \frac{f ( t )}{S ( t )} .$$

$$\text{?} \text{indicates text missing or illegible when filed}$$

Reasoning about the intensity function instead of the density allows for the specification of well-established self-excitation processes, such as the Hawkes process. In the general case, with a parametric form of the intensity specified, maximum likelihood estimation is possible but can involve certain challenges. The likelihood is as follows: Σᵢ₌₁^{N} log p_{θ}*(tᵢ)=Σᵢ₌₁^{N} log λ₀*(tᵢ)−∫₀^{t}^{_{N}}λ₀*(s)ds as shown in Rasmussen (2018). The difficulty may arise in choosing a flexible parametric specification for the intensity function that still has a closed form integral. Shchur et al. (2019) address this issue by directly modeling p*_{θ}(t) in the setting where times and events are considered independently.

### Exemplary Multivariate Temporal Point Process

A multivariate temporal point process can be defined as a random process that is used to model event streams. An event stream can be a sequence of N events {(tᵢ, eᵢ)}ᵢ₌₁^{N} where tᵢ is the time that the ith event occurs and eᵢ∈E is the event type chosen from a set of possible events E. A key characteristic of a truly multivariate point process is that the events are tightly coupled with the times. This dependence may be characterized by the conditional intensity function for each event

$${λ}_{e}^{*} ( t ) = {lim}_{Δ t \to 0} \frac{\text{?}}{Δ t} P ( t \leq T < t + Δ t , E = e \vert T \geq t , ℋ )$$

$$\text{?} \text{indicates text missing or illegible when filed}$$

which is also known as a cause-specific hazard function. This hazard function can represent the instantaneous rate at which a given event is happening in the presence of competing events. The hazard functions for each event can specify a joint likelihood over the entire sequence which can be derived as follows:

$${λ}^{*} ( t ) = \text{?} ( t ) \qquad ( 1 )$$

$$P ( {T}_{i} \leq t \vert ℋ ) = 1 - exp ( - \text{?} {{{\sum }^{\vert ε \vert}}_{e = 1}}_{}^{} {λ}_{e}^{*} ( t ) dt )$$

$$P ( {T}_{i} = t \vert ℋ ) = exp ( \text{?} {{{\sum }^{\vert ε \vert}}_{e = 1}}_{}^{} {λ}_{e}^{*} ( t ) dt ) * \text{?}$$

$$P ( {E}_{i} = e \vert {T}_{i} = t , ℋ ) = \frac{{λ}_{e}^{*} ( t )}{{\sum }_{e = 1}^{\vert ε \vert} \text{?} ( t )}$$

$$P ( {T}_{i} = t , {E}_{i} = e \vert ℋ ) = {λ}_{e}^{*} ( t ) * exp ( - \text{?} {{{\sum }^{\vert ε \vert}}_{e = 1}}_{}^{} {λ}_{e}^{*} ( t ) ) dt )$$

$$\text{?} \text{indicates text missing or illegible when filed}$$

Prior approaches which may model the conditional intensity functions, thus, do incorporate competing risks of all the events but at the cost of the necessity to take a Monte Carlo estimate of the integral in the second term of the objective function. This specification may also complicate the sampling process as it typically requires a thinning algorithm.

### Exemplary Popcorn Model

### Exemplary Construction of Objective

The exemplary system, method and computer-accessible medium, according to an exemplary embodiments of the present disclosure, can be called POint Processes for Competing Observations with Recurrent Networks, or POPCORN. In the exemplary system, method and computer-accessible medium, according to an exemplary embodiments of the present disclosure, instead of modeling the conditional intensity, one can directly model the conditional probability of each event given history p*_{ϵ}(t)=p_{ϵ}(t|H). This can be distinct from the joint probability in (1) which is often labeled in a similar way as in (Enguehard et al., 2020). The exemplary model can make the assumption that the conditional probabilities of each of the event time distributions are conditionally independent given history.

The exemplary system, method and computer-accessible medium, according to an exemplary embodiments of the present disclosure, can offer several advantages relative to directly modeling the conditional probabilities. These exemplary advantages can include the ability to directly sample and a simple, closed-form likelihood, while maintaining flexibility by using a mixture density network to model each conditional probability.

In exemplary embodiments of the present disclosure, pₑ*(t) can be the conditional probability given history, Sₑ*(t) can be the survival function given history and hₑ*(t) can be the hazard function given history.

$$P ( {T}_{i} = t , {E}_{i} = \text{?} \vert ℋ ) = P ( {T}_{i} = t \vert ℋ ) P ( E = e \vert {T}_{i} = t , H ) \qquad ( 2 )$$

$$P ( {T}_{i} \leq t \vert ℋ ) = 1 - {\prod }_{e = 1}^{\vert ε \vert} {S}_{e}^{*} ( t )$$

$$\begin{matrix}P ( {T}_{i} = t \vert ℋ ) = ( {\prod }_{e = 1}^{\vert ε \vert} {S}_{e}^{*} ( t ) ) ( {{\sum }^{\vert ε \vert}}_{e = 1} \frac{{p}_{e}^{*} ( t )}{{S}_{e}^{*} ( t )} ) \\ = ( {\prod }_{e = 1}^{\vert ε \vert} {S}_{e}^{*} ( t ) ) ( {{\sum }^{\vert ε \vert}}_{e = 1} \text{?} ( t ) )\end{matrix}$$

$$P ( {E}_{i} = e \vert {T}_{i} = t , ℋ ) ∝ \text{?} {\prod }_{j \neq e} {S}_{j}^{*} ( t )$$

$$P ( {T}_{i} = t , {E}_{i} = e \vert ℋ ) = \frac{{p}_{e}^{*} ( t ) \text{?} {S}_{j}^{*} ( t )}{{\sum }_{e = 1}^{\vert ε \vert} {p}_{e}^{*} ( t ) \text{?} {S}_{j}^{*} ( t )} ( {\prod }_{e = 1}^{\vert ε \vert} {S}_{e}^{*} ( t ) ) ( {{\sum }^{\vert ε \vert}}_{e = 1} \text{?} ( t ) )$$

$$\text{?} \text{indicates text missing or illegible when filed}$$

Assuming that the conditional probability and the survival function can be computed, this likelihood can be closed-form and stochastic optimization can be conducted. By modeling each of the conditional probabilities separately, the exemplary embodiments can sample from this model simply by taking a sample from all of the event distributions and taking the minimum time as the next time and event.

### Exemplary Sampling and Connection to Competing Risk

In competing risk problems, an idea is that there are latent or potential failure times T₁, . . . Tₑ. A multiple decrement, or joint survival function, can be described as follows where z is a feature vector and we have e different event types:

$$Q ( {t}_{1} , … {t}_{e} ; z ) = P ( {T}_{1} > {t}_{1} , … {T}_{e} > {t}_{e} , z ) \qquad ( 3 )$$

In this setting, the data which is observed can be described in the following way:

$$T = min \{ {T}_{1} , … {T}_{e} \} , \qquad ( 4 )$$

$$E = \{ j \vert {T}_{j} \leq {T}_{k} , k = 1 … e \}$$

This can extend to the setting of point processes but the interpretation can become that there is a separate competing risk problem for each timestep for a given patient. Exemplary embodiments of the present disclosure can directly specify this joint survival function because the exemplary embodiment models the probabilities of each event separately given history and assume conditional independence. This means that the joint survival function can simply be the product of each of the survival distributions of the conditional probability densities.

Thus, sampling can be straightforward: (1) Sample from each of the conditional distributions to get a set of t₁ . . . t_{|E|} and take the minimum; and/or (2) This minimum can provide both the time until the next event and the event itself.

### 4.1 Exemplary Conditional Independence Assumption and Identifiability

The assumption of conditional independence may at first appear restrictive. However, as Tsiatis (1975) shows given any joint survival function with arbitrary dependencies between events, there can exist a different joint function which is specified by independent risks that models the data just as precisely. This result can make it very difficult or even impossible to test whether competing risks are independent. This theorem can show that given there is a sufficiently flexible way of modeling each conditional distribution, exemplary embodiments can recover an equivalent model to any model which incorporates dependent risks.

### Exemplary Mixture Density Networks and Distributional Specifications

In order to specify a flexible distributional specification for each of the conditional probabilities, exemplary embodiments can use mixture density networks. In particular, exemplary embodiments can use a mixture of Weibull distributions and a mixture of Fréchet distributions for all our experiments.

Exemplary Mixture of Weibulls: The exemplary Weibull distribution is a common distribution for specifying survival in survival analysis because its parameters have a direct interpretation. It can have a shape (k) and a scale (l) parameter, where the shape parameter controls whether the hazard is increasing or decreasing overtime. Thus, a mixture of Weibulls could capture the combination of many different possible hazard shapes. Exemplary embodiments can use an MLP to generate parameters for the Weibull and the mixture weights (w) from the historical encoding. A Softplus transform can be used to ensure that the parameters are restricted to positive real numbers and weights are normalized. The pdf for a mixture of Weibulls is the following:

$$p ( t ; l , k , w ) = {\sum }_{i = 1}^{J} {w}_{i} \frac{{k}_{i}}{{l}_{i}} {( \frac{t}{{l}_{i}} )}^{{k}_{i} - 1} exp ( - {( \frac{t}{{l}_{i}} )}^{{k}_{i}} ) \qquad ( 5 )$$

$$( k , l , w ) = {MLP}_{θ} ( {h}_{t} ) \qquad ( 6 )$$

Exemplary Mixture of Fréchets: The exemplary Fréchet distribution is also known as the Inverse Weibull and can have similar properties to the Weibull in that it can be defined on the positive reals, can have shape (α) and scale (s) parameters and can have a favorable form for the pdf and cdf which make it amenable for likelihood-based optimization. One distinction is that the Fréchet may have heavy tails which can make it more stable for optimization purposes and more robust to outliers in the data. Exemplary embodiments define this mixture in a similar way:

$$p ( t ; s , α , w ) = {\sum }_{i = 1}^{J} {w}_{i} \frac{{α}_{i}}{{s}_{i}} ( \frac{t}{{s}_{i}} \text{?} exp ( - ( \frac{t}{{s}_{i}} \text{?} ) \qquad ( 7 )$$

$$( α , s , w ) = {MLP}_{θ} ( {h}_{t} ) \qquad ( 8 )$$

$$\text{?} \text{indicates text missing or illegible when filed}$$

Exemplary Encoding History with Multi-Channel LSTM

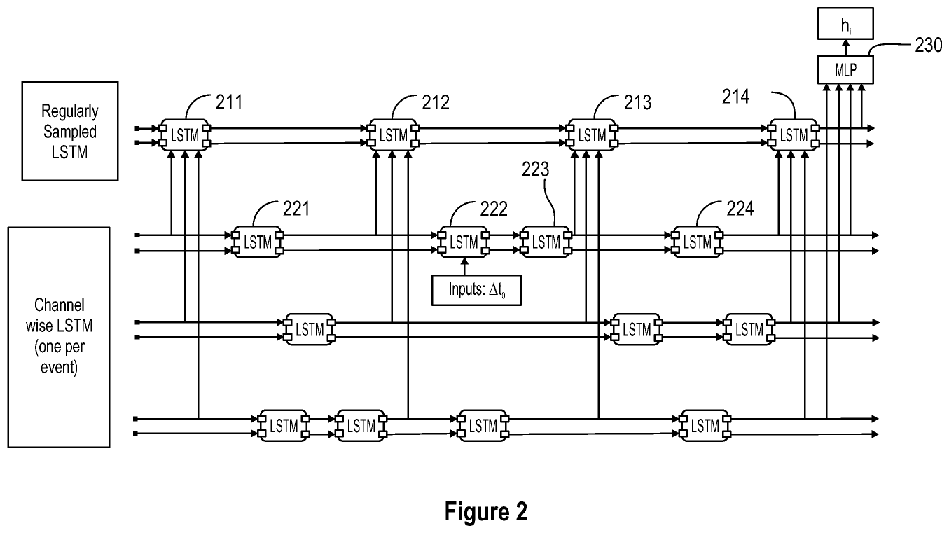

FIG. 2 shows an exemplary Multi-Channel LSTM diagram for encoding history according to exemplary embodiments of the present disclosure. For example, each event may have its own distinct LSTM which can keep track of the progression of interevent times for that particular event. The regularly-sampled LSTM periodically can collect the hidden states of all other channels to model dependencies across the channels overtime. At any given time, exemplary embodiments may then collect all most recent hidden states, concatenate them and encode them as a single vector representing history.

In order to encode history, exemplary embodiments use a multi-channel LSTM architecture which is shown in the exemplary FIG. 2. Each event may have its own dedicated LSTM which captures its irregular dynamics. The inputs to each LSTM (e.g., LSTM 221, 222, 223, and 224) can be the time differences since the last observation of the event Δt=t_{e,i}−t_{e,i-1} where t_{j,i} represents the absolute time of the ith observation of the jth event. Additionally, exemplary embodiments can have an LSTM channel dedicated to modeling dependencies across the rest of the channels over time. This LSTM (e.g., LSTM 211, LSTM 212, LSTM 213, LSTM 214) can take as input the concatenated hidden states from each of the event-specific channels at a regular interval which is pre-specified. At each subsequent timestep, exemplary embodiments can then extract all the hidden states of each LSTM and the regularly-sampled LSTM at that specific time and concatenate them. Exemplary embodiments can use them as input into an MLP 230 to create a hidden encoding of the history.

An exemplary motivation, among many others, behind using such an approach is to capture the nature of the irregular sampling for each event. Additionally, such an approach may mitigate the problem of vanishing gradients especially for events which are rarely observed.

### Exemplary Handling Multiple Events at a Given Time

In general, multiple events at a given time are assumed to never occur in the context of point processes. EHR data, however, can contain events which have the same timestamp largely as a function of documentation practices. Exemplary embodiments of the present disclosure can handle this by adjusting the exemplary objective function to allow a subset of the events to occur at a given time. Exemplary embodiments can use an indicator vector to represent which events are present and which are absent.

The multi-label objective can be expressed as follows:

$$( 9 )$$

$$\begin{matrix}P ( {T}_{i} = t , {E}_{i} = \text{?} ) = ( {\prod }_{e = 1}^{\vert ε \vert} P ( {E}_{i} = e \vert {T}_{i} = t \text{?} ( 1 - P ( {E}_{i} = e \vert {T}_{i} = t ) \text{?} ) \\ P ( \text{?} = t ) \\ = \text{?} P ( {E}_{i} = e , {T}_{i} = t \text{?} ( P ( {T}_{i} = t ) - P ( {E}_{i} = e , {T}_{i} = t ) \text{?} \text{?}\end{matrix}$$

$$\text{?} \text{indicates text missing or illegible when filed}$$

This can be, perhaps effectively, converting what was a categorical cross entropy to a binary cross entropy. Enguehard et al. (2020) constructs a similar loss for this situation. However, in their loss they are not modeling a conditional probability as we are but rather a joint probability as in Equation 1. They, instead, construct the following likelihood:

$$( 10 )$$

$$P ( {T}_{i} = t , {E}_{i} = \text{?} ) = {\prod }_{e = 1}^{\vert ε \vert} P ( {E}_{i} = e , {T}_{i} = t \text{?} ( 1 - min ( P ( {E}_{i} = e , {T}_{i} = t \text{?} , 1 ) \text{?}$$

$$\text{?} \text{indicates text missing or illegible when filed}$$

This exemplary likelihood assumes that the joint density can be treated as discrete and can be constrained to be between 0 and 1. Thus, it may require bounding the joint density to compute. Due to this discrepancy and the lack of bound on the first term, it is difficult to compare the exemplary models on the NLL metric.

The general framework of point processes may not allow for simultaneous events. This likelihood provides an exemplary simple approach towards doing so. Notably, there is related research (see Solo, 2007) on how to handle ties in a more principled fashion.

TABLE 1
Exemplary Dataset Description

| | | | Avg. | | | |
|---|---|---|---|---|---|---|
| Dataset | Events | Task Type | Length | Train | Val | Test |
| MIMIC-II | 75 | Multi-class | 4 | 585 | 65 | 65 |
| Stack Overflow | 22 | Multi-class | 72 | 5307 | 1326 | 1326 |
| Ear Infection | 39 | Multi-label | 2 | 8179 | 1022 | 1023 |
| Synthea Full | 357 | Multi-label | 43 | 10524 | 585 | 585 |

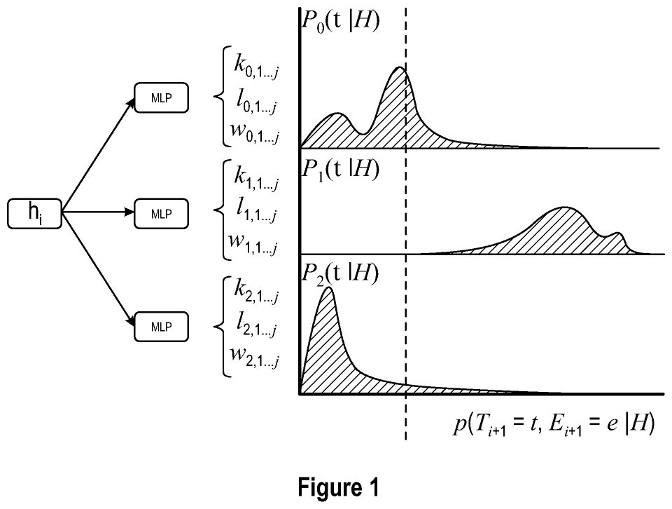

FIG. 1 shows high level diagram and associated graphs for an exemplary POPCORN model according to an exemplary embodiment of the present disclosure. In this example embodiment, the hidden encoding of history is mapped via MLPs to Weibull mixture parameters (mixture density network) and this can result in various conditional multi-modal distributions which are then used to compute the objective.

### Exemplary Experiments

### Exemplary Datasets

Exemplary experiments can be run on four datasets in total. These may be the same datasets and dataset splits that were used in the work most closely related to the present disclosure (see Enguehard et al. (2020)). This exemplary experiment can use both common benchmarks used in the point process community and synthetic EHR data to encourage transparency and reproducibility. This also allows the exemplary embodiment to compare reported metrics directly.

Exemplary MIMIC-II: This can be a dataset that has been used for benchmarking point processes methods in numerous past works. It consists of a sequence of hospital visits where each event is a different disease diagnosis. The average length of each sequence can be relatively small (4) making this less of a longitudinal dataset than the full Synthea dataset.

Exemplary Stack Overflow: This dataset represents two years of user awards on a question-answering website. Each event is a user receiving a badge (of 22 different types) and when they received this badge. Although this dataset is not health related, it is used in almost every other point process paper as a benchmark and as such we used it to test the generalizability of the exemplary model.

Exemplary Synthea: Ear Infection: This dataset is simulated based upon the Synthea (Walonoski et al., 2018) EHR simulator which leverages a Markov process with several states informed by the input of human experts and population summary statistics. There are several modules in this simulator—this dataset leverages the Ear Infection module which is a simplified version of the full simulator that contains patients who experience ear infections. It consists of encounter types, conditions and medications associated with ear infections and any comorbidities associated with age of onset. This dataset can be a simplified version of the full EHR simulation which has clear dependencies between time and next event.

Exemplary Synthea, Full Simulation: The full Synthea simulation consists of much longer longitudinal sequences (on average 43) of encounters, conditions and medications administered. Some of the most frequent events in this dataset include ER admission, viral sinusitis, insulin administration, and prenatal visits (among the 357 different event types).

### Exemplary Metrics

Exemplary F1 and AUROC: In order to evaluate how well the exemplary model does on next-event prediction, exemplary embodiments use a weighted F1 score in the multi-class case (where only a single event can be observed at a given time) and weighted AUROC in the multi-label case (where multiple events can be observed at a given time). It should be noted that this is next-event prediction conditioned on the next time (as has been conventionally reported in past work).

TABLE 2
Exemplary Hyperparameter Settings for Reported Models

| | Batch | | No. | Hidden | Hidden |
|---|---|---|---|---|---|
| Dataset | Size | Distribution | Mix | Enc | LSTM |
| MIMIC-II | 16 | Weibull | 2 | 16 | 8 |
| Stack Overflow | 32 | Fréchet | 4 | 16 | 8 |
| Ear Infection | 16 | Fréchet | 2 | 16 | 8 |
| Synthea Full | 16 | Weibull | 4 | 16 | 8 |

Exemplary Negative Log Likelihood: Exemplary embodiments additionally report Negative Log Likelihood (NLL) normalized by time for the multi-class datasets (as this metric may not directly be comparable with baselines for multi-label cases, see Section 4.6). The NLL can be a measure of how well the model is capturing both time and event.

### Exemplary Hyperparameters

Exemplary embodiments list the most important hyperparameter settings in Table 2 which include batch sizes, distributional specification, number of mixture components, hidden embedding size and hidden size inside the channel LSTMs. Exemplary embodiments use the Adam optimizer with a learning rate of 1e-3 for all runs, running every model for 100 epochs with early stopping criteria based on validation NLL.

### Exemplary Results and Discussion

Exemplary Findings: Performance on the metrics is shown in Table 3 and Table 4 aggregated across five different splits, with sample standard deviation values over the splits in parenthesis. The results show that the exemplary model is able to achieve strong performance across all the datasets, particularly on next-event prediction. The exemplary model is compared to 4 baselines which are reported in Enguehard et al. (2020): Conditional Poisson (CP), RMTPP (see Du et al., 2016), a Log Normal Mixture model (see Shchur et al., 2019) and the best performing NeuralTPP model (see Enguehard et al., 2020) for each dataset.

For the multi-class problems, the exemplary model performs competitively on F1 and NLL/time, achieving a better F1 score on the MIMIC-II dataset. For the multi-label case, the exemplary model performs equally well on AUROC on the Synthea Ear Infection dataset and significantly better on the full Synthea dataset over all baselines. As mentioned before, it may not be possible to directly compare the exemplary results on the NLL/time metric as the likelihood functions are not exactly the same.

The exemplary model outperforms CP, RMTPP and the LogNormMix on next-event prediction for all tasks. All of these baselines consider time and event independently. This shows that for EHR data, among others, incorporating this dependence can important. Furthermore, the assumption of conditional independence of event time distributions does not constrain model performance on next-event prediction as compared to the Neural TPP approaches which consider dependent competing risks. This provides some empirical evidence that for a sufficiently flexible specification of conditional distributions, EHR data can be, e.g. effectively, modeled despite this assumption.

TABLE 3
Exemplary Results on MIMIC and Stack Overflow

| | MIMIC-II | | Stack Overflow | |
|---|---|---|---|---|
| Model | F1 Score | NLL/time | F1 Score | NLL/time |
| CP | .691 (.083) | 6.78 (1.99) | .325 (.004) | .553 (.003) |
| RMTPP | .709 (.076) | 4.24 (2.66) | .284 (.004) | .592 (.006) |
| LogNorm Mix | .705 (.170) | 6.33 (.370) | .314 (.003) | .548 (.004) |
| Neural TPP | .648 (.098) | 4.61 (2.49) | .342 (.006) | .543 (.005) |
| POPCORN | .772 (.046) | 5.07 (1.17) | .330 (.005) | .542 (.003) |
| (Ours) | | | | |

TABLE 4
Exemplary Results on Synthea Datasets

| | Synthea (Ear Infection) | Synthea (Full) |
|---|---|---|
| Model | AUROC Score | AUROC Score |
| CP | .792 (.009) | .850 (.014) |
| RMTPP | .675 (.068) | .616 (.043) |
| LogNorm Mix | .767 (.007) | .770 (.010) |
| Neural TPP | .857 (.005) | .822 (.006) |
| POPCORN (Ours) | .853 (.008) | .886 (.008) |

### Exemplary AUROC by Event Type

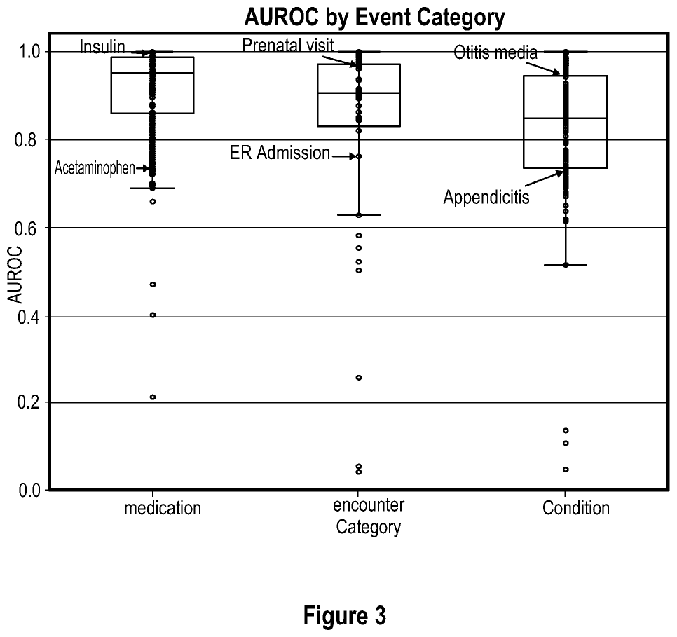

FIG. 3 shows an exemplary diagram for visualizing AUROC by event type accordingly to an exemplary embodiment of the present disclosure.

For example, in order to examine which events the model is predicting best on the Synthea (Full) dataset, exemplary embodiments visualize AUROC by event type illustrated in FIG. 3. At a higher level, the exemplary model is able to predict medications most easily while conditions are more difficult. In particular, for conditions which are potentially less predictable such as a concussion or appendicitis, the exemplary model may not perform as well. Medications which are commonly administered or prescribed for specific diseases (e.g. insulin for diabetes or furosemide for heart disease) are easier for the model to predict.

Exemplary Model Performance by Length of History: In order to evaluate the exemplary performance over long sequences of longitudinal data, the present disclosure investigated how AUROC varied as a function of the number of observations seen on the Synthea (Full) dataset. Table 5 shows that that without any history and for shorter sequences, prediction may be more difficult. The longer the sequence, the more dependencies are able to be learned overtime. After collecting enough data about a particular patient's history (between 10-20 observations), the exemplary model is better able to reason about what comorbidities and medications a patient likely has and is likely to have in the future. The assumption of conditional independence is also mitigated by the collection of more history which shows that for longer longitudinal sequences, such an assumption may be reasonable.

TABLE 5
Exemplary Performance by Sequence History Length on Synthea (Full)

| Sequence Interval | 0-1 | 0-5 | 0-10 | 0-20 | 0-30 | 0-40 | 0-50 | 0-60 | 0-70 | 0-80 | 0-90 |
|---|---|---|---|---|---|---|---|---|---|---|---|
| AUROC | .601 | .648 | .723 | .807 | .835 | .848 | .857 | .864 | .869 | .872 | .876 |

### Exemplary Practical Applications Embodiments

### Causal Inference

Exemplary Sepsis interventions: Sepsis is a condition which occurs as a result of the body's reaction to infection. It can cause a cascade of organ failures and is potentially fatal. It is a condition where the early signs and symptoms may not be easily discernible yet early intervention can be crucial for better outcomes. Some exemplary interventions can include fluids and vasopressors. Often it may not be clear which interventions to take at what time. An exemplary trained POPCORN model according to exemplary embodiments of the present disclosure can assess the causal effect of one intervention versus another based on some favorable future outcome (e.g., lower likelihood of death, delay in sepsis etc.) by, e.g., simulating many different future trajectories. This can provide the clinician with, e.g., guidance on which intervention can produce better outcomes. The POPCORN model can provide personalized recommendations based on individual, patient-level causal effect estimates.

Comparative effect estimation of multiple drugs (e.g., diabetes, blood thinners): There can be two drugs commonly used to treat a disease, yet it is not clear whether one drug should be favored over another for certain subsets of patients. An exemplary trained POPCORN model according to exemplary embodiments of the present disclosure can be deployed to assess future trajectories of individual patients for each drug and provide a ranking for the drug that can produce the best outcomes. This exemplary scenario occurs in, e.g., blood pressure reduction medications and diabetes blood sugar management.

### Exemplary Multi-Step Prediction:

Exemplary Sepsis prediction: Applying the exemplary POPCORN model to this inpatient setting can address the irregular sampling of lab tests in the EHR, encoding of patient history and model multiple outcomes (e.g., death, sepsis, septic shock etc.). The exemplary model according to exemplary embodiments of the present disclosure can predict the risk of occurrence of sepsis, and thus, enabling interventions where necessary.

Coronary artery disease (CAD)/Stroke risk assessment: CAD and stroke are both common conditions where risk accumulates overtime (e.g., age, diet, smoking, physical activity over a lifetime). The exemplary POPCORN model can model data over many inpatient and outpatient visits and handle longer-term dependencies in the dataset which can affect risk assessment. The exemplary model would be able to predict time-to-event of stroke/CAD and assign risk scores to different patients to help doctors make interventions where needed.

### Simulation of Synthetic Data:

Simulating synthetic data for sensitive populations (hemophilia, HIV): In many cases, due to privacy concerns, data on sensitive populations is not available for public research use. An exemplary POPCORN model trained with data from these sensitive populations can simulate fully synthetic EHR (or claims) datasets which can be made available to the broader research community.

### Further Exemplary Description

The exemplary system, method and computer-accessible medium, according to an exemplary embodiments of the present disclosure, can be a multivariate point process model for EHR data which can have, among others, a number of advantages: (1) it can specify a dependence between event and time; (2) it can allow for direct sampling; and/or (3) it can specify a closed-form likelihood, making optimization straightforward. Exemplary embodiments of the present disclosure demonstrate that the exemplary approach matches or outperforms baseline approaches on the task of next-event prediction on all three clinical datasets. In particular, exemplary embodiments of the present disclosure outperform all baselines which do not take dependence between event and time into account for prediction. This dependence, while may be less important in certain datasets, can be considered when modeling EHR data. Results also show that the exemplary model, which assumes conditional independence of event time distributions, performs similarly or better than NeuralTPP, as expected based on the theoretical results of Tsiatis (1975). Given the significant advantages (such as, for example, direct sampling and closed-form likelihood) that such an assumption enables, the exemplary approach should be strongly considered when such properties are particularly desirable. Certain embodiments can investigate different methods of handling ties which may reflect more closely the reality of the documentation process, evaluate the exemplary model on real longitudinal EHR data, and explore related applications such as encoding latent representations of history. Furthermore, exemplary embodiments can evaluate the exemplary approach's ability to generate realistic samples of data and its performance on time-to-event with alternative metrics.

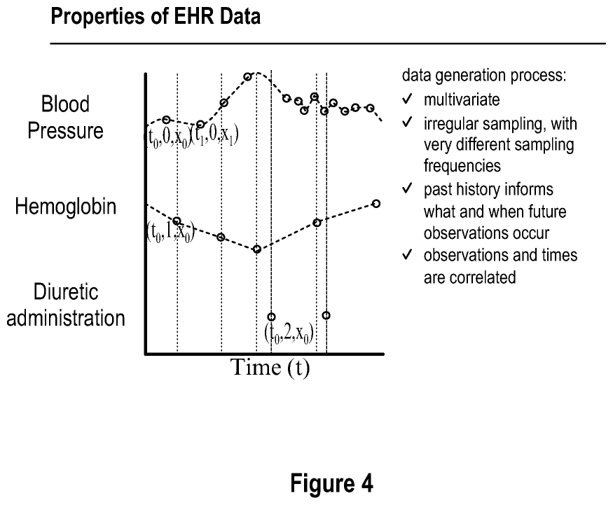

FIG. 4 shows an exemplary diagram of synthetic data in the EHR which may be represented as a multivariate point process accordingly to an exemplary embodiment of the present disclosure. In the diagram, three different features collected during a patient stay: blood pressure, hemoglobin, and diuretic administration are shown. After blood pressure goes up, e.g., a diuretic is administered and BP goes back to normal and hemoglobin goes up. Additionally, the sampling frequency of both hemoglobin and BP changes as physicians more closely monitor the patient.

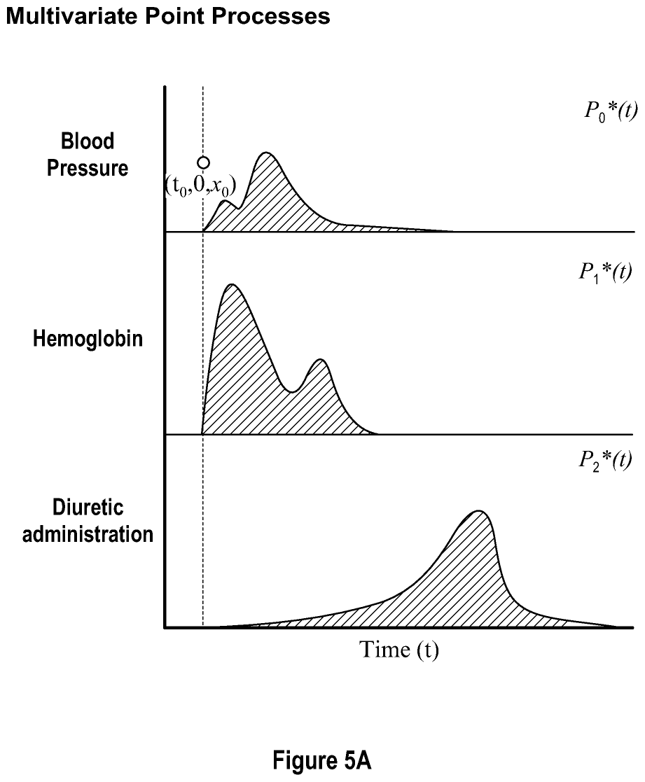

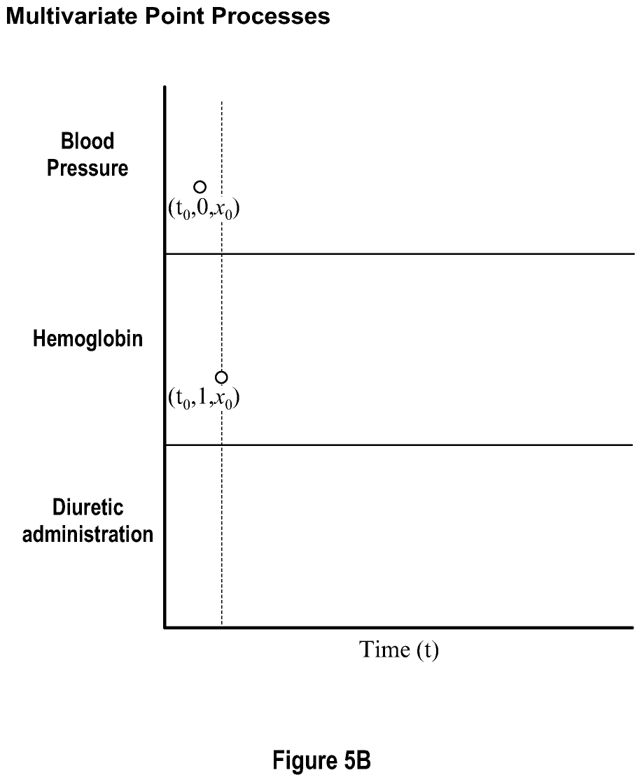

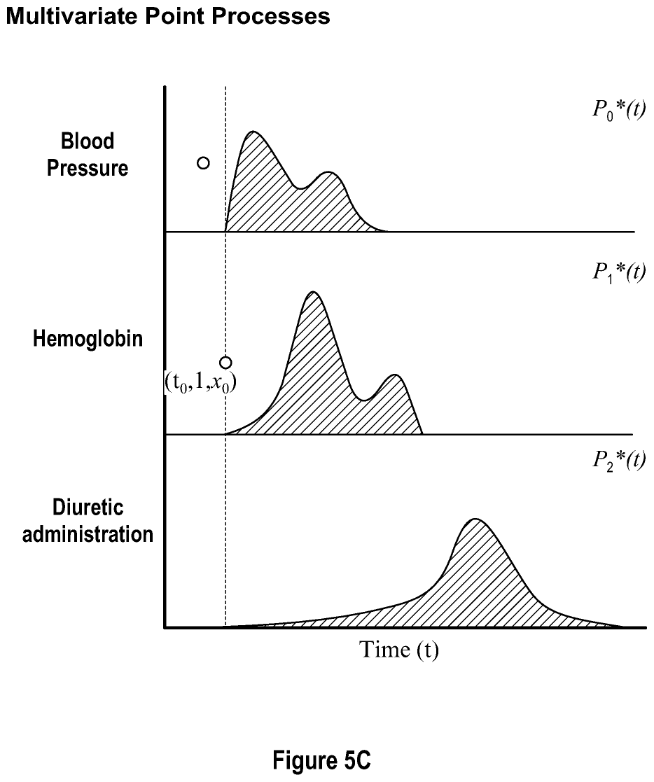

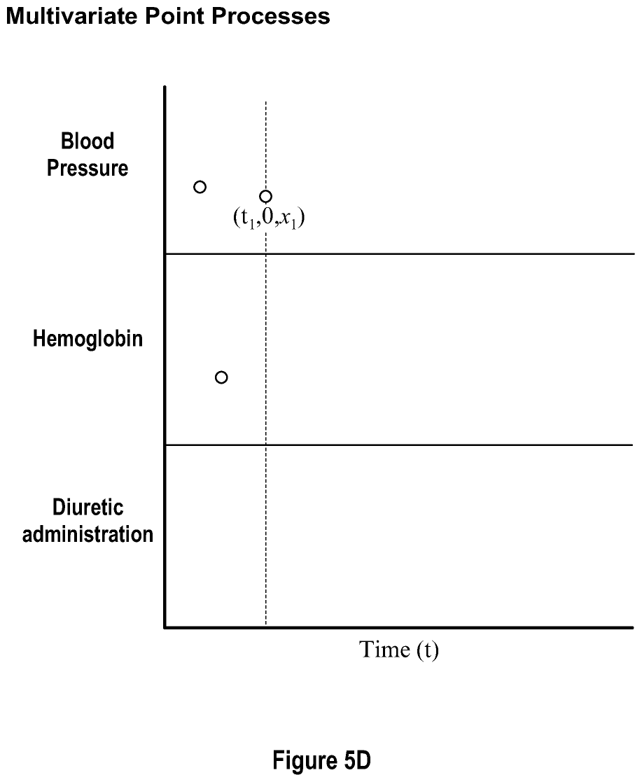

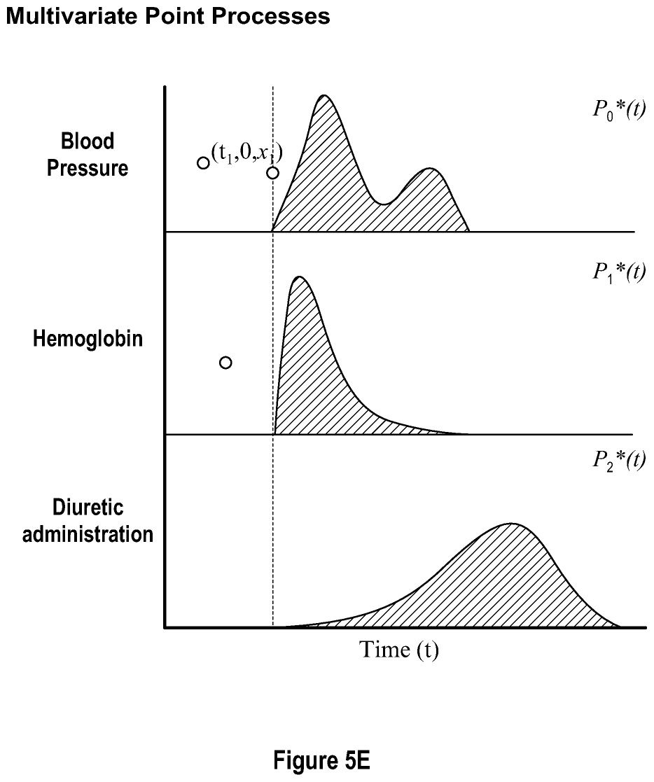

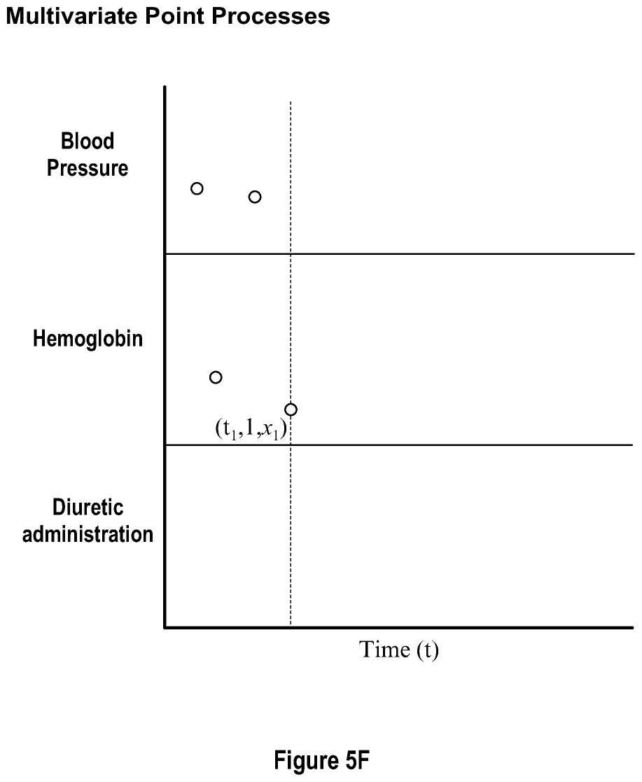

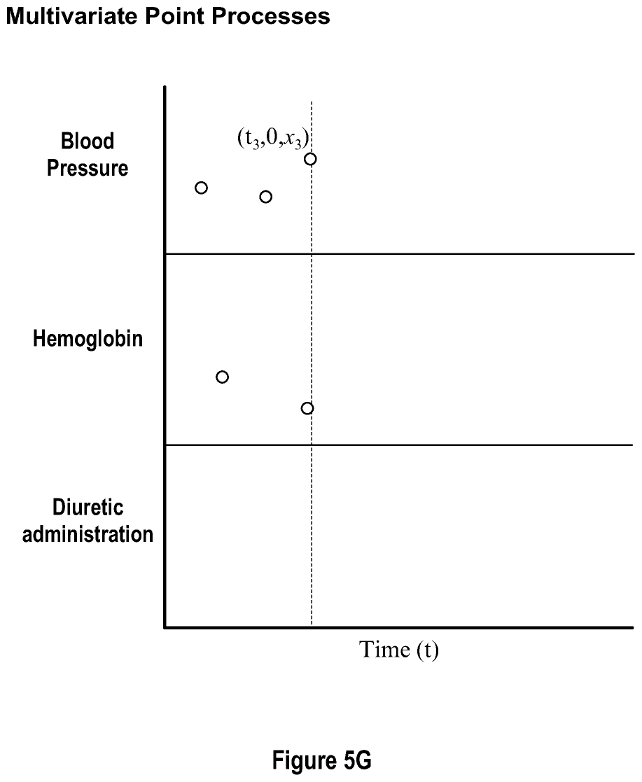

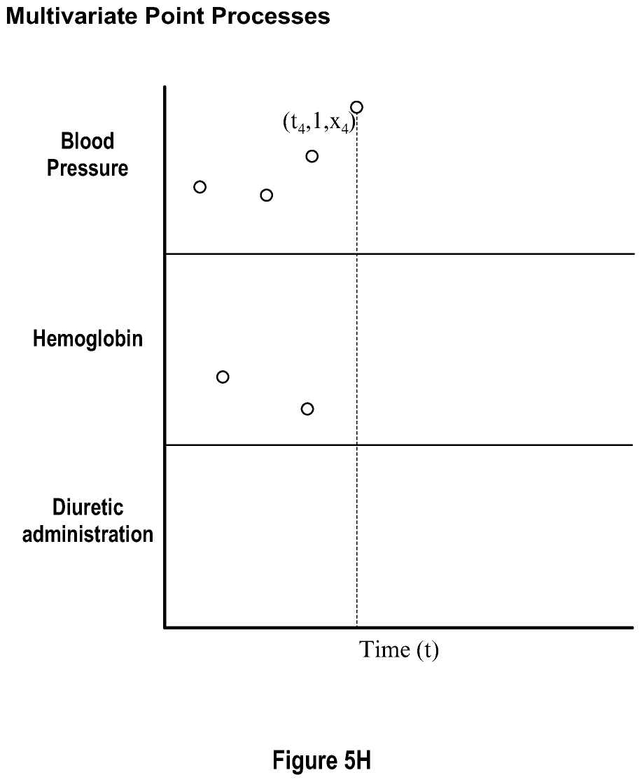

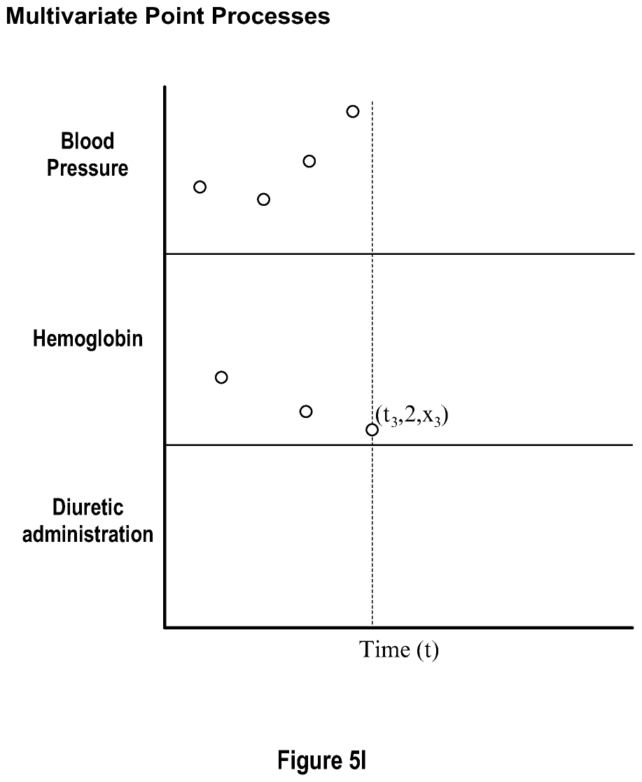

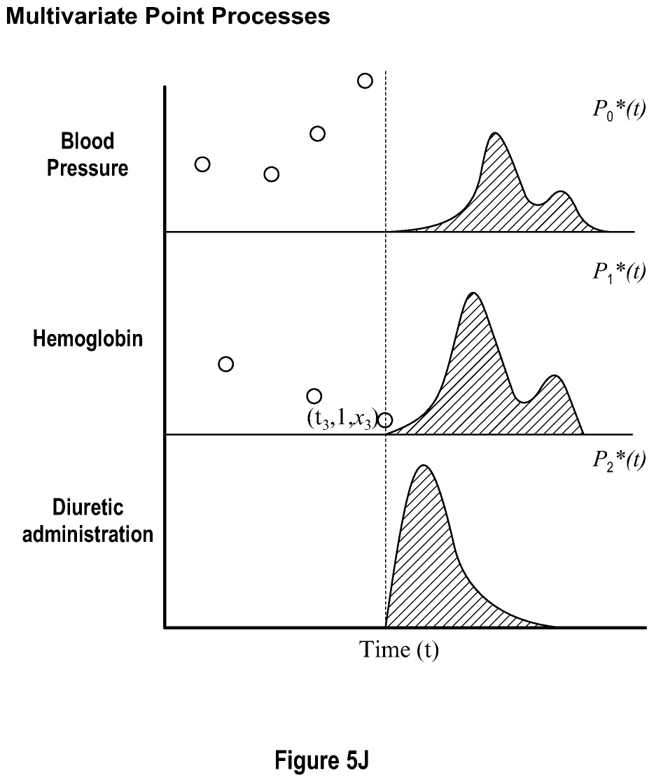

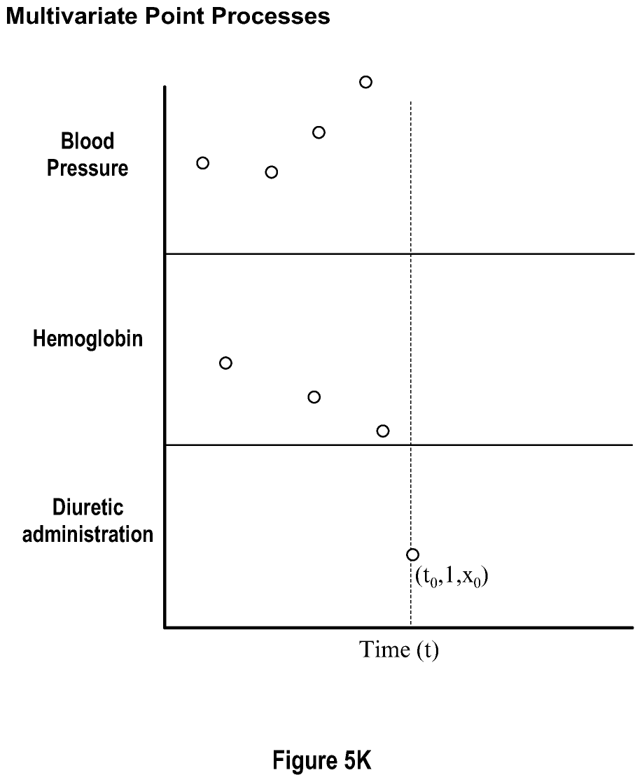

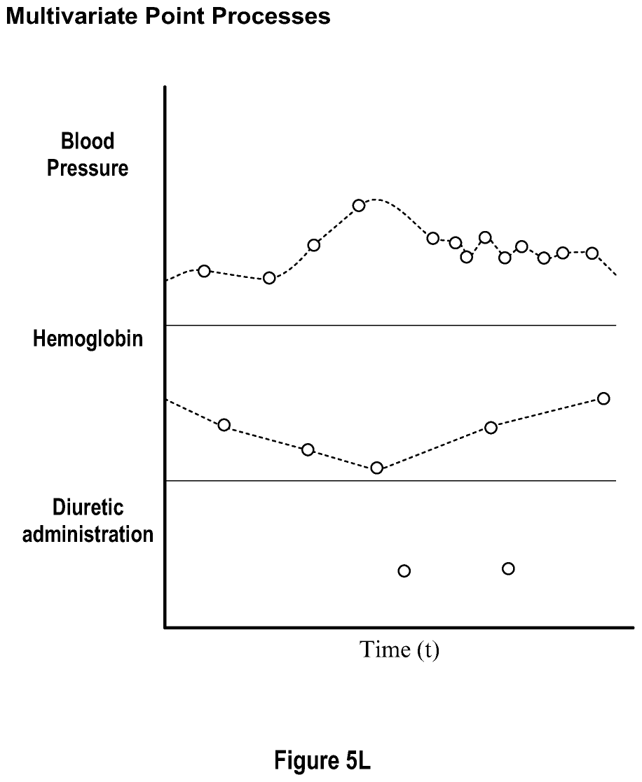

FIGS. 5(a)-5(l) show exemplary diagrams and illustrations of an exemplary multivariate point process as it is sampled forward according to an exemplary embodiment of the present disclosure. In each successive figure, a new point is sampled based on the predicted event probability distributions for time-to-event. For example, such new point can be subsequently incorporated into the history and the next event distributions are obtained. In this way, a full longitudinal sample is obtained.

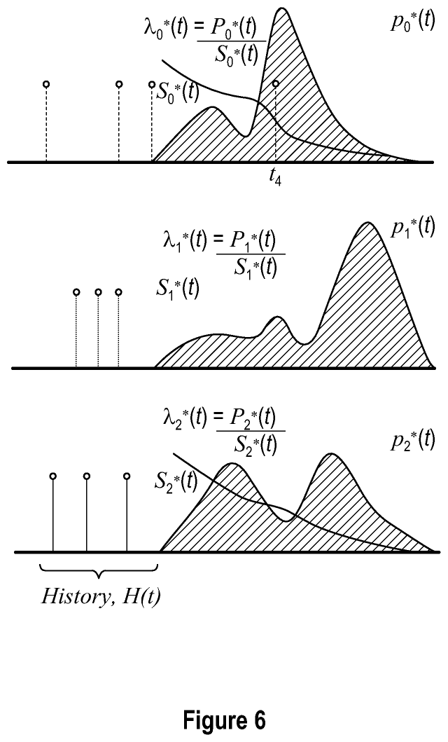

FIG. 6 shows an exemplary illustration of an exemplary multivariate point process which is modeled in a more traditional manner using conditional intensity functions Ai rather than event probabilities.

### Exemplary Baselines

For example, all the baselines compared against the exemplary embodiments are described in detail in Enguehard et al. (2020), the entire disclosure of which is incorporated by reference. These basslines are described as follows.

Exemplary Conditional Poisson The conditional poisson model assumes that the event intensities are constant overtime (and thus assumes exponential event distributions specified by a parameter). This model also assumes that the next event and next time are conditionally independent.

$${λ}^{*} ( t ) = MLP ( {h}_{t} ) \qquad ( 11 )$$

The MLP can take the historical encoding and transform it to a constant which is then used to specify the closed-form likelihood.

Exemplary RMTPP The RMTPP model (Du et al., 2016) may use the following specification:

$${λ}^{*} ( t ) = exp ( \text{?} {h}_{j} + {w}^{t} ( t - {t}_{j} ) + \text{?} ) \qquad ( 12 )$$

$$P ( {y}_{j + 1} = k \vert {h}_{j} ) \frac{exp ( \text{?} {h}_{j} + {b}_{k}^{y} )}{{\sum }_{k = 1}^{K} exp ( \text{?} {h}_{j} + {b}_{k}^{y} )} \qquad ( 13 )$$

$$\text{?} \text{indicates text missing or illegible when filed}$$

where v^{t}(column vector), w^{t}(scalar), b^{t}(scalar) and V^{y} (matrix of size k by |hⱼ|) and b^{y} are all parameters of the model.

Additionally, hⱼ can be the historical encoding which they obtain using an RNN which takes in tuples of the historical sequence.

Such a model may have a more complicated intensity function than a conditional poisson but still requires the intensity to have an exponential formulation which results in a closed-form Gompertz likelihood. This model also models next events and times independently as shown above.

Exemplary Log Normal Mixture The Log Normal Mixture model (Shchur et al., 2019) can use a mixture distribution to directly model the event distribution as follows:

$$p ( τ \vert w , μ , s ) ] = {\sum }_{k = 1}^{K} {w}_{k} \frac{1}{τ {s}_{k} \sqrt{2 π}} exp ( - \frac{{( log τ - {μ}_{k} )}^{2}}{2 {s}_{k}^{2}} ) \qquad ( 14 )$$

where w are the mixture weights, μ are the mixture means and s are the standard deviations.

These mixture weights can be parameterized by an embedding of past history as follows:

$${w}_{i} = softmax ( {V}_{w} {h}_{i} + {b}_{w} )$$

and sᵢ=exp(Vₛhᵢ+bₛ) {V_{w}, Vₛ, V_{μ}, b_{w}, bₛ, b_{μ}} are learnable parameters. The μᵢ=V_{μ}hᵢ+b_{μ} next event can be modeled independently:

$${π}_{i} = softmax ( {V}_{π}^{( 2 )} tanh ( {V}_{π}^{( 1 )} {h}_{i} + {b}_{π}^{( 1 )} ) + {b}_{π}^{( 2 )} ) \qquad ( 15 )$$

where {V_{π}⁽¹⁾, V_{π}⁽²⁾, b⁽¹⁾_{π}, b⁽²⁾_{π}} are parameters of the network and πᵢ is the categorical probabilities over the next events.

In order to encode history, they use the same architecture as RMTPP. One of the advantages of this model over RMTPP is that it has a much more flexible distribution for the intensity (and thus for the event distributions).

Exemplary Neural TPPs The Neural TPP models are a class of models which specify an encoder, decoder architecture. The encoder architecture encodes past history into a hidden vector and the decoder architecture specifies either (1) an analytical conditional intensity function for each event or (2) a cumulative conditional intensity function for each event. Within this framework, they have 2 encoder architectures and 4 decoder architectures which can be used interchangeably. For the encoders, they use either a standard GRU network or a Self-Attention (SA) network. For the decoder networks, they use either MLPs or attention networks to generate a conditional intensity or cumulative intensity. Additional details are provided at the appendix of Enguehard et al. (2020).

### EXEMPLARY Synthea Dataset Details

Exemplary Synthea Ear Infection This dataset can be simulated based upon the Synthea (Walonoski et al., 2018) EHR simulator which leverages a Markov process with several states informed by the input of human experts and population summary statistics. The ear infection module consists of encounter types, conditions and medications associated with ear infections and any comorbidities associated with age of onset. Table 6 shows all possible encounters, conditions, and/or medications that are in this dataset along with their relative counts in a single fold of the training data.

Exemplary Synthea Full Dataset The full Synthea simulation consists of much longer longitudinal sequences (on average 43) of encounters, conditions and medications administered. Table 7 includes the exemplary top 10 event names, types, codes and relative counts within each event category for a single fold of the training data.

### Exemplary Code

Exemplary code for the POPCORN model and exemplary data is provided at the following link: https://github.com/abhave77/POPCORN, the entirety of which is incorporated by reference in this disclosure.

TABLE 6
List of all possible events in Ear Infection dataset with event types and codes

| Event Name | Event Category | Event Code | Count |
|---|---|---|---|
| Encounter for symptom | encounter | SNOMED-CT 185345009 | 10282 |
| Otitis media | condition | SNOMED-CT 65363002 | 10282 |
| Acetaminophen 160 MG Chewable Tablet | medication | RxNorm 313820 | 4384 |
| Amoxicillin 250 MG Oral Capsule | medication | RxNorm 308182 | 2992 |
| Aspirin 81 MG Oral Tablet | medication | RxNorm 243670 | 2972 |
| Ibuprofen 100 MG Oral Tablet | medication | RxNorm 198405 | 2217 |
| Penicillin G 375 MG/ML Injectable Solution | medication | RxNorm 105078 | 1713 |
| Doxycycline Monohydrate 50 MG Oral Tablet | medication | RxNorm 1652673 | 912 |
| Cefuroxime 250 MG Oral Tablet | medication | RxNorm 309097 | 871 |
| General examination of patient (procedure) | encounter | SNOMED-CT 162673000 | 755 |
| Ampicillin 100 MG/ML Injectable Solution | medication | RxNorm 789980 | 734 |
| Cefaclor 250 MG Oral Capsule | medication | RxNorm 309045 | 645 |
| Clopidogrel 75 MG Oral Tablet | medication | RxNorm 309362 | 590 |
| Nitroglycerin 0.4 MG/ACTUAT Spray | medication | RxNorm 705129 | 424 |
| Amoxicillin 500 MG Oral Tablet | medication | RxNorm 308192 | 406 |
| Coronary Heart Disease | condition | SNOMED-CT 53741008 | 360 |
| Simvastatin 20 MG Oral Tablet | medication | RxNorm 312961 | 348 |
| Acetaminophen 325 MG Oral Tablet | medication | RxNorm 313782 | 347 |
| Amlodipine 5 MG Oral Tablet | medication | RxNorm 197361 | 341 |
| Stroke | condition | SNOMED-CT 230690007 | 307 |
| Alteplase 100 MG Injection | medication | RxNorm 1804799 | 270 |
| 1 ML Epinephrine 1 MG/ML Injection | medication | RxNorm 1660014 | 265 |
| Atropine Sulfate 1 MG/ML Injectable Solution | medication | RxNorm 1190795 | 265 |
| Cardiac Arrest | condition | SNOMED-CT 410429000 | 265 |
| History of cardiac arrest (situation) | condition | SNOMED-CT 429007001 | 257 |
| 3 ML Amiodarone hydrocholoride 50 MG/ML | medication | RxNorm 834357 | 251 |
| Warfarin Sodium 5 MG Oral Tablet | medication | RxNorm 855332 | 211 |
| Digoxin 0.125 MG Oral Tablet | medication | RxNorm 197604 | 211 |
| Verapamil Hydrochloride 40 MG | medication | RxNorm 897718 | 210 |
| Ibuprofen 200 MG Oral Tablet | medication | RxNorm 310965 | 202 |
| Atrial Fibrillation | condition | SNOMED-CT 49436004 | 202 |
| Well child visit (procedure) | encounter | SNOMED-CT 410620009 | 173 |
| Naproxen sodium 220 MG Oral Tablet | medication | RxNorm 849574 | 160 |
| Myocardial Infarction | condition | SNOMED-CT 22298006 | 144 |
| History of myocardial infarction (situation) | condition | SNOMED-CT 399211009 | 134 |
| Captopril 25 MG Oral Tablet | medication | RxNorm 833036 | 128 |
| Atorvastatin 80 MG Oral Tablet | medication | RxNorm 259255 | 104 |
| 12 HR Cefaclor 500 MG Oral Tablet | medication | RxNorm 309043 | 55 |
| Doxycycline Monohydrate 100 MG Oral Tablet | medication | RxNorm 1650142 | 48 |

TABLE 7
List of top 10 most frequent events by category in
Synthea (Full) dataset with event types and codes

| Event Name | Event Category | Event Code | Count |
|---|---|---|---|
| Viral sinusitis (disorder) | condition | SNOMED-CT 444814009 | 32379 |
| Acute viral pharyngitis (disorder) | condition | SNOMED-CT 195662009 | 19169 |
| Normal pregnancy | condition | SNOMED-CT 72892002 | 16233 |
| Acute bronchitis (disorder) | condition | SNOMED-CT 10509002 | 15901 |
| Otitis media | condition | SNOMED-CT 65363002 | 8710 |
| Streptococcal sore throat (disorder) | condition | SNOMED-CT 43878008 | 5616 |
| Sprain of ankle | condition | SNOMED-CT 44465007 | 3641 |
| Anemia (disorder) | condition | SNOMED-CT 271737000 | 2880 |
| Body mass index 30+ - obesity (finding) | condition | SNOMED-CT 162864005 | 2750 |
| Prediabetes | condition | SNOMED-CT 15777000 | 2062 |
| Encounter for symptom | encounter | SNOMED-CT 185345009 | 89739 |
| General examination of patient (procedure) | encounter | SNOMED-CT 162673000 | 72374 |
| Encounter for check up (procedure) | encounter | SNOMED-CT 185349003 | 23610 |
| Consultation for treatment | encounter | SNOMED-CT 698314001 | 23390 |
| Emergency room admission (procedure) | encounter | SNOMED-CT 50849002 | 22673 |
| Prenatal initial visit | encounter | SNOMED-CT 424441002 | 16233 |
| Follow-up encounter | encounter | SNOMED-CT 390906007 | 13545 |
| Encounter for problem | encounter | SNOMED-CT 185347001 | 11072 |
| Encounter Inpatient | encounter | SNOMED-CT 183452005 | 7911 |
| Well child visit (procedure) | encounter | SNOMED-CT 410620009 | 5988 |
| Hydrochlorothiazide 25 MG Oral Tablet | medication | RxNorm 310798 | 27383 |
| insulin human isophane 70 UNT/ML | medication | RxNorm 106892 | 20105 |
| amLODIPine 5 MG | medication | RxNorm 999967 | 17760 |
| Acetaminophen 325 MG Oral Tablet | medication | RxNorm 313782 | 17173 |
| 24 HR Metformin hydrochloride 500 MG | medication | RxNorm 860975 | 17170 |
| Atenolol 50 MG Oral Tablet | medication | RxNorm 746030 | 16524 |
| NDA020503 200 ACTUAT Albuterol 0.09 MG | medication | RxNorm 2123111 | 14255 |
| 120 ACTUAT Fluticasone propionate 0.044 MG | medication | RxNorm 895994 | 14255 |
| Simvastatin 10 MG Oral Tablet | medication | RxNorm 314231 | 12214 |
| Hydrochlorothiazide 12.5 MG | medication | RxNorm 429503 | 10509 |

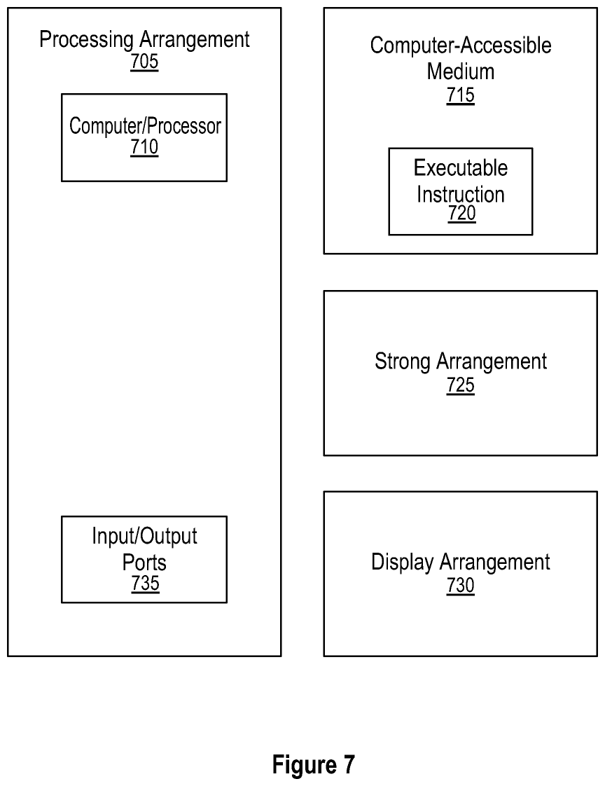

FIG. 7 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As illustrated in FIG. 7, for example, a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 so as to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the internet, an intranet, a data collection probe, a sensor, etc. As shown in FIG. 7, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
1. Zhengping Che, Sanjay Purushotham, Kyunghyun Cho, David Sontag, and Yan Liu. Recurrent neural networks for multivariate time series with missing values. Scientific reports, 8(1):1-12, 2018.
2. Nan Du, Hanjun Dai, Rakshit Trivedi, Utkarsh Upadhyay, Manuel Gomez-Rodriguez, and Le Song. Recurrent marked temporal point processes: Embedding event history to vector. In Proceedings of the 22nd ACM SIGKDD International Conference on Knowledge Discovery and Data Mining, pages 1555-1564, 2016.
3. Joseph Enguehard, Dan Busbridge, Adam Bozson, Claire Woodcock, and Nils Hammerla. Neural temporal point processes for modelling electronic health records. In Machine Learning for Health, pages 85-113. PMLR, 2020.
4. Alistair E W Johnson, Tom J Pollard, Lu Shen, Li-wei H Lehman, Mengling Feng, Mohammad Ghassemi, Benjamin Moody, Peter Szolovits, Leo Anthony Celi, and Roger G Mark. Mimic-iii, a freely accessible critical care database. Scientific data, 3:160035, 2016.
5. Hongyuan Mei and Jason Eisner. The neural hawkes process: A neurally self-modulating multivariate point process. arXiv preprint arXiv:1612.09328, 2016.
6. Maya Okawa, Tomoharu Iwata, Takeshi Kurashima, Yusuke Tanaka, Hiroyuki Toda, and Naonori Ueda. Deep mixture point processes: Spatio-temporal event prediction with rich contextual information. In Proceedings of the 25th ACMSIGKDD International Conference on Knowledge Discovery & Data Mining, pages 373-383, 2019.
7. Takahiro Omi, Naonori Ueda, and Kazuyuki Aihara. Fully neural network based model for general temporal point processes. arXiv preprint arXiv:1905.09690, 2019.
8. Jakob Gulddahl Rasmussen. Lecture notes: Temporal point processes and the conditional intensity function. arXiv preprint arXiv:1806.00221, 2018.
9. Oleksandr Shchur, Marin Bilŏs, and Stephan Günnemann. Intensity-free learning of temporal point processes. arXiv preprint arXiv:1909.12127, 2019.
10. Victor Solo. Likelihood functions for multivariate point processes with coincidences. In 2007 46th IEEE Conference on Decision and Control, pages 4245-4250. IEEE, 2007.
11. Behzad Tabibian, Isabel Valera, Mehrdad Farajtabar, Le Song, Bernhard Schölkopf, and Manuel Gomez-Rodriguez. Distilling information reliability and source trustworthiness from digital traces. In Proceedings of the 26th International Conference on World Wide Web, pages 847-855, 2017.
12. Matthew A Taddy, Athanasios Kottas, et al. Mixture modeling for marked poisson processes. Bayesian Analysis, 7(2):335-362, 2012.
13. Anastasios Tsiatis. A nonidentifiability aspect of the problem of competing risks. Proceedings of the National Academy of Sciences, 72(1):20-22, 1975.
14. Jason Walonoski, Mark Kramer, Joseph Nichols, Andre Quina, Chris Moesel, Dylan Hall, Carlton Duffett, Kudakwashe Dube, Thomas Gallagher, and Scott McLachlan. Synthea: An approach, method, and software mechanism for generating synthetic patients and the synthetic electronic health care record. Journal of the American Medical Informatics Association, 25(3):230-238, 2018.

1. A method for predicting medical events used for a treatment of at least particular one of a plurality of patients, comprising:
receiving first medical information for each of the patients, wherein the medical information includes at least one of the medical events and a time associated with the at least one of the medical events;
generating a summary of the medical information;
generating a multivariate point process model based on the summarized medical information, wherein a computation of a non-estimated probability distribution is used to train the multivariate point process model;
receiving second medical information for the at least particular one of the patients; and
predicting and facilitating at least possible one of the medical events and a predicted time of the at least possible one of the medical events for the at least particular one of the patients.

2. The method of claim 1, further comprising specifying a dependence between the future time and the future event.

3. The method of claim 1, wherein the multivariate point process model specifies a conditional probability of each of the medical events.

4. The method of claim 3, wherein the multivariate point process model is based on a survival function and a history function which is associated with the summarized medical information.

5. The method of claim 4, wherein the conditional probability is determined based on the survival function in view of the history function.

6. The method of claim 1, wherein the multivariate point process model facilitates a generation of the at least possible one of the medical events and the predicted time based on a sample from all of event distributions.

7. The method of claim 1, further comprising facilitating or controlling the treatment of the at least particular one of the patients based on the generated at least possible one of the medical events and the predicted time.

8. A method for predicting medical events used for a treatment of at least particular one of a plurality of patients, comprising:
receiving first medical information for each of the patients, wherein the medical information includes at least one of the medical events and a time associated with the at least one of the medical events;
generating a summary of the medical information;
generating a multivariate point process model based on the summarized medical information, wherein each of the medical events has its own distinct sub-model which tracks progression of interevent times for that particular medical event;
receiving second medical information for the at least particular one of the patients; and
predicting and facilitating at least possible one of the medical events and a predicted time of the at least possible one of the medical events for the at least particular one of the patients.

9. The method of claim 8, further comprising specifying a dependence between the future time and the future event.

10. The method of claim 8, wherein the multivariate point process model specifies a conditional probability of each of the medical events.

11. The method of claim 10, wherein the multivariate point process model is based on a survival function and a history function which is associated with the summarized medical information.

12. The method of claim 11, wherein the conditional probability is determined based on the survival function in view of the history function.

13. The method of claim 8, wherein the multivariate point process model facilitates a generation of the at least possible one of the medical events and the predicted time based on a sample from all of event distributions.

14. The method of claim 8, further comprising facilitating or controlling the treatment of the at least particular one of the patients based on the generated at least possible one of the medical events and the predicted time.

15. A non-transitory computer-accessible medium having stored thereon computer-executable instructions for determining phenotypic information for a treatment of at least particular one of a plurality of patients, the computing arrangement is configured to perform procedures comprising:
receiving first medical information for each of the patients, wherein the medical information includes at least one of the medical events and a time associated with the at least one of the medical events;
generating a summary of the medical information;
generating a multivariate point process model based on the summarized medical information, wherein a computation of a non-estimated probability distribution is used to train the multivariate point process model;
receiving second medical information for the at least particular one of the patients; and
predicting and facilitating at least possible one of the medical events and a predicted time of the at least possible one of the medical events for the at least particular one of the patients.

16-21. (canceled)

22. A non-transitory computer-accessible medium having stored thereon computer-executable instructions for determining phenotypic information for a treatment of at least particular one of a plurality of patients, the computing arrangement is configured to perform procedures comprising:
receiving first medical information for each of the patients, wherein the medical information includes at least one of the medical events and a time associated with the at least one of the medical events;
generating a summary of the medical information;
generating a multivariate point process model based on the summarized medical information, wherein each of the medical events has its own distinct sub-model which tracks progression of interevent times for that particular medical event;
receiving second medical information for the at least particular one of the patients; and
predicting and facilitating at least possible one of the medical events and a predicted time of the at least possible one of the medical events for the at least particular one of the patients.

23-28. (canceled)

29. A system for predicting medical events used for a treatment of at least particular one of a plurality of patients, comprising:
a computer hardware arrangement configured to:
receive first medical information for each of the patients, wherein the medical information includes at least one of the medical events and a time associated with the at least one of the medical events;
generate a summary of the medical information;
generate a multivariate point process model based on the summarized medical information, wherein a computation of a non-estimated probability distribution is used to train the multivariate point process model;
receive second medical information for the at least particular one of the patients; and
predict and facilitate at least possible one of the medical events and a predicted time of the at least possible one of the medical events for the at least particular one of the patients.

30-34. (canceled)

35. The system of claim 29, wherein the computer hardware arrangement is further configured to facilitate or control the treatment of the at least particular one of the patients based on the generated at least possible one of the medical events and the predicted time.

36. A system for predicting medical events used for a treatment of at least particular one of a plurality of patients, comprising:
a computer hardware arrangement configured to:
receive first medical information for each of the patients, wherein the medical information includes at least one of the medical events and a time associated with the at least one of the medical events;
generate a summary of the medical information;
generate a multivariate point process model based on the summarized medical information, wherein each of the medical events has its own distinct sub-model which tracks progression of interevent times for that particular medical event;
receive second medical information for the at least particular one of the patients; and
predict and facilitate at least possible one of the medical events and a predicted time of the at least possible one of the medical events for the at least particular one of the patients.

37-41. (canceled)

42. The system of claim 36, wherein the computer hardware arrangement is further configured to facilitate or control the treatment of the at least particular one of the patients based on the generated at least possible one of the medical events and the predicted time.
